(19) United States
(12) Patent Application Publication
(10) Pub. No.: US 2025/0017690 A1
(43) Pub. Date: Jan. 16, 2025

(54) METHOD FOR COLLECTING USAGE DATA OF AN ENDODONTIC INSTRUMENT

(71) Applicants: Walid NEHME, BEIRUT (LB); MICRO-MEGA INTERNATIONAL, BESANCON (FR)

(72) Inventors: Walid NEHME, BEIRUT (LB); Stéphane CLAUDE, LA ROCHE (CH)

(73) Assignees: Walid NEHME, BEIRUT (LB); MICRO-MEGA INTERNATIONAL, BESANCON (FR)

(21) Appl. No.: 18/717,508

(22) PCT Filed: Sep. 27, 2022
(86) PCT No.: PCT/FR2022/051825

§ 371 Date: Jun. 7, 2024

(30) Foreign Application Priority Data
Dec. 9, 2021 (FR) 2113223

(51) Int. Cl. A61C 5/48; A61C 19/04

(52) U.S. Cl. CPC A61C 5/48; A61C 19/042

(57) ABSTRACT

A method for collecting usage data of an endodontic instrument. The method includes the following steps: acquiring data relating to the shape of a root canal to be treated; at regular intervals, acquiring data relating to the working depth of the instrument in the root canal; at regular intervals, acquiring data relating to the stress undergone by the instrument during work; and producing a database linking each data item acquired.

### TECHNICAL FIELD

The invention relates to the technical field of endodontics.

### PRIOR ART

The endodontic treatment of a root canal consists of evacuating the tissues from that canal. To perform that operation, the practitioner uses root canal instruments, such as exploration files, in order to identify the trajectory of the canal, determine specifically the length of the canal, then evacuate the tissues using another file before shaping the canal with a view to its filling.

During an endodontic treatment, instrument breakage is one of the most common complications. The direct complications of such instrument breakage are of a clinical nature:
if it is possible to recover the broken instrument, the consequences are an increase in the treatment duration and potential weakening of the tooth by a reduction of thickness of the residual wall necessary to remove the broken instrument;
if it is not possible to recover the broken instrument, the consequences can (i) be linked to insufficient disinfection of the root canal system entailing post-operative pain, failure to heal, and therefore failure of the endodontic treatment, or (ii) be linked to the onset of an infectious process not existing at the time and resulting in pain and failure of the treatment in the medium term.

Occurrences of instrument breakage are generally reduced by the design of root canal instruments, in order to make them more resistant to the stresses exerted during endodontic treatment.

However, instrument breakage is a rapid event, which can occur in less than a few seconds, and it is difficult to know the conditions under which the breakage occurred.

Several parameters can influence the occurrence of a breakage, such as:
the shape of the canal treated, particularly if it presents a complex shape, with a sudden change of direction forming a sharp bend;
the irrigation of the canal, which may or may not be implemented by the practitioner during certain phases of the treatment;
the effort the practitioner exerts on the instrument.

The possibilities for improvement of the design of the instruments are therefore limited by the lack of knowledge of these multiple parameters.

It is also difficult, even for an experienced practitioner, to know precisely how to manipulate an instrument within the canal during treatment. Even if the practitioner knows that the canal he is going to treat is complex, and considers he is taking all the necessary precautions, he may be mistaken and not correctly adapt his approach or the dynamic drive of the instrument to the complexity of the canal, which can lead to instrument breakage.

### PRESENTATION OF THE INVENTION

The aim of the invention is to make up for the drawbacks of the prior art, by proposing a method for collecting usage data of endodontic instruments in order to best know the conditions leading to instrument breakage.

For that purpose, a method has been devised for collecting usage data of an endodontic instrument.

According to the invention, the method comprises the following steps:
acquiring data relating to the shape of a root canal to be treated;
at regular intervals, acquiring data relating to the working depth of the instrument in the root canal;
at regular intervals, acquiring data relating to the stress undergone by the instrument during use;
producing a database linking each data item acquired.

In that way, it is possible to determine to what stresses the instrument was subject for each position it occupied within the canal. The database can be made available to manufacturers of instruments or handpieces designed to drive the instruments, such that they have much fuller information about the conditions of use of the equipment, and the design and/or conditions of use can be adapted with the aim of reducing the occurrences of breakage.

Excess stress exerted by the practitioner is detectable, for example.

It is also possible to observe that certain shapes of canals, previously considered to be non-critical, in reality induce stresses on the instrument which are far higher than that expected.

In order to keep the database to a limited size, the data are acquired at regular intervals, and the interval is a predefined length, for example every 0.5 mm. In that way, it is possible to know the evolution of the stresses for each position of the instrument within the canal, and therefore in each form that the canal imposed on the instrument.

In order that the database is as exhaustive as possible, the data are acquired at regular intervals according to a predefined duration, for example 0.5 s. In that way, it is possible to know the evolution of the position and of the stresses over time, for the whole duration of the treatment.

For the acquisition to be as complete as possible, and best reflect the reality of the approach accomplished, the data relating to the shape is acquired in three dimensions.

Preferably, the method also collects and saves data of the instrumental dynamic within the database. Knowing the instrument dynamic, for example the speed of rotation, makes it possible to have more information in order to perform the desired analyses. With that same aim, additional data, such as the status of activation or inactivation of the irrigation, are also recorded within the database.

So as to be able to target more specifically the treatments having led to a breakage, the additional data include information on whether or not the instrument breaks during the treatment.

For the data collected to be as exhaustive as possible, the data relating to stress are a complete torsor at a given point of the stress experienced by the instrument, obtained by measuring the stress according to three axes of an orthonormal reference point.

Advantageously, the method includes a step of finite-element calculation based on:
the shape of the canal;
a shape and the mechanical properties of the instrument;
the working depth of the instrument at a given interval or given moment; and
the stress at that same given interval or given moment;
in order to determine the local stresses within a material constituting the instrument for recording at the given interval. It is thus possible to detect zones where stresses are concentrated within the instrument, using real data, with a view to best adapting the design of the instrument and/or its planned conditions of use, such as the instrument dynamic to be adopted.

The invention also concerns a handpiece for the endodontic practice, comprising a control unit running a computer program, and designed to drive a root canal instrument.

According to the invention, the handpiece comprises means for detecting the distance between a reference point and a part of a tooth, and means for measuring the mechanical stresses experienced by the instrument, connected to the control unit. The computer program is configured to determine a depth at which the instrument is during use according to a known length of the instrument, and to determine the mechanical stresses experienced by the instrument during use, and the computer program is configured to upload to a database the data acquired relative to the mechanical stresses and the depth (Lp).

Such handpiece therefore comprises all structural elements making it possible to implement the method according to the aforementioned features, with the resulting advantages.

### BRIEF DESCRIPTION OF THE DRAWINGS

FIG. 1 is a diagram of a tooth, illustrating the shape of a canal to be treated.

FIG. 2 is a diagram of a root canal instrument.

FIG. 3 is an illustration of a handpiece according to the invention.

FIG. 4 is a diagram illustrating such handpiece during use.

FIG. 5 is a diagram of a treatment in a first configuration.

FIG. 6 is a diagram of a finite-element calculation in that first configuration.

FIG. 7 is a diagram of the treatment in a second configuration.

FIG. 8 is a diagram of a finite-element calculation in that second configuration.

FIG. 9 is a diagram illustrating a database categorising different shapes of root canals.

### DETAILED DESCRIPTION OF THE INVENTION

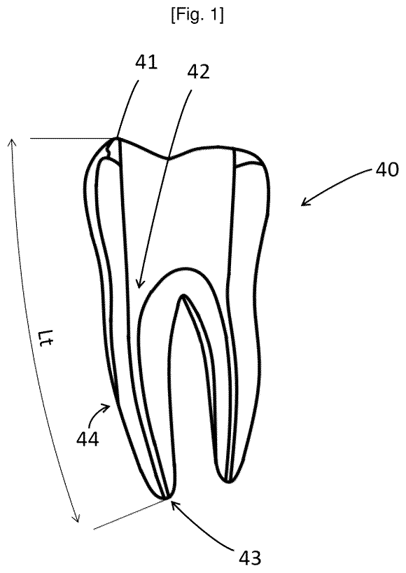

In reference to FIG. 1, an endodontic treatment consists of removing the tissue from a root canal (42) of a tooth (40). At the time of that treatment, an instrument (20), such as a file, should be used up to the apex (43) of the canal (42). The length between a part (41) of the tooth (40), generally a canine, and the apex (43) is the working length (Lt).

As the canal (42) is roughly filiform, the lengths are measured according to the dimensions of the canal (42). However, the canal (42) can present different types of shape, which has an influence over the complexity of the treatment to be performed.

A canal (42) can be straight, in which case the treatment will be simple. Conversely, a canal (42) presenting a curve (44) will exert stress on the instrument (20). In the extreme case where that curve (44) takes the form of a sharp bend, the level of stress is such that it can lead to breakage of the instrument (20).

It is therefore common to obtain an acquisition of the shape of the canal (42) to be treated, for example by x-ray, in order that the practitioner can best anticipate his approach, and can predict which are the zones where the instrument (20) is at risk of breaking.

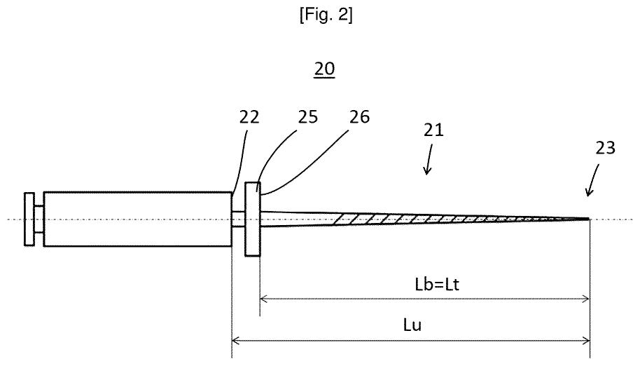

In reference to FIG. 2, an instrument (20) is generally endowed with a buffer (25) made of an elastomer material. The position of that buffer is adjusted by the practitioner so the buffer length (Lb), defined by the distance between the tip (23) of the instrument (20) and an underside (26) of the buffer (25), is equal to the working length (Lt). The buffer length (Lb) is therefore less than or equal to a usable length (Lu) of the instrument (20).

In that way, when the practitioner performs his treatment and penetrates the canal (42) with the instrument (20), he knows that when the underside (26) reaches the contact of the part (41), then the tip (23) is at the level of the apex (43). However, while the underside (26) is not in contact with the part (41), the practitioner has no precise information about the position of the instrument (20) within the canal (42), such that the instrument (20) can reach a curve (44) or a high-risk zone, when the practitioner has not prepared for that.

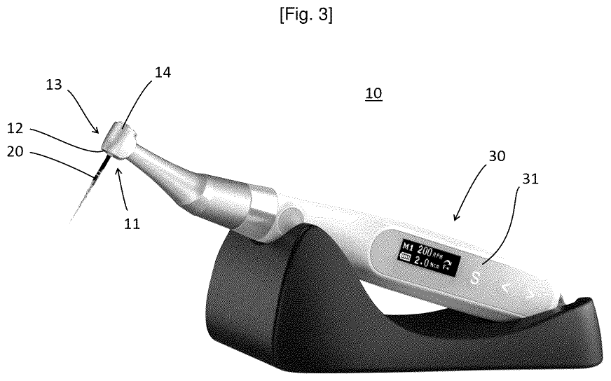

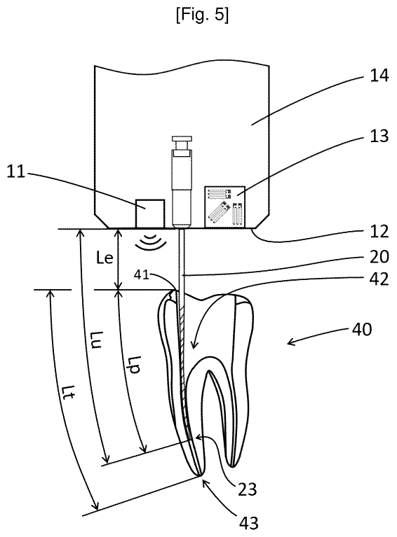

In reference to FIGS. 3 to 5, the handpiece (10) according to the invention comprises a control unit (30) running a motor designed to drive an instrument (20) according to an appropriate dynamic, via a counter-angle (14).

The handpiece (10) comprises a depth gauge (11) making it possible to know in real time the working depth (Lp) of the instrument within the canal (42). The depth gauge (11) comprises means of detection which measure a distance (Le) between the part (41) of the tooth (40) and the reference point (12) which is preferably on the handpiece (10).

Deducting the distance (Le) from the usable length (Lu) provides the working depth (Lp) at the instrument (20) is at a given moment.

Using the acquisition of the shape of the canal (42) and the working depth (Lp) at a given moment, it is possible to know whether the tip (23) is preparing to approach a high-risk zone (44). More generally, it is also possible to reconstruct the shape presented by the instrument (20) at that given moment, since it is modelled by the trajectory of the canal (42).

The handpiece (10) also comprises means (13) for measuring the mechanical stresses experienced by the instrument (20). The means (13) for measuring are of any appropriate type, and can be dynamometers or extensometers. Preferably they are strain gauges, for example axial or in the form of rosettes. Indeed, these strain gauges are small, durable, and their output signal is easy to interpret.

In addition to the cutting forces, the stresses experienced by the instrument (20) derive from the efforts that the practitioner applies to the instrument (20) upon treatment, if he pushes more or less with the handpiece (10), and aligned with the canal (42) or not.

The distribution of those efforts within the instrument (20) depends of course on the shape of the instrument (20), but also on the shape of the canal (42), so the form it imposes on the instrument (20), which presents a certain flexibility. If the canal (42) presents a sharp bend, then the instrument (20) is also bent. If the instrument (20) is driven in rotation, it is then subject to stresses of alternating flexion, which the instrument (20) does not experience in a zone where the canal (42) is straight.

The distribution of the efforts experienced by the instrument (20) makes it possible to achieve the distribution of the stresses within it.

In order that the measurements are as accurate as possible, the depth gauge (11) and the means (13) for measuring are preferably arranged against the counter-angle (14).

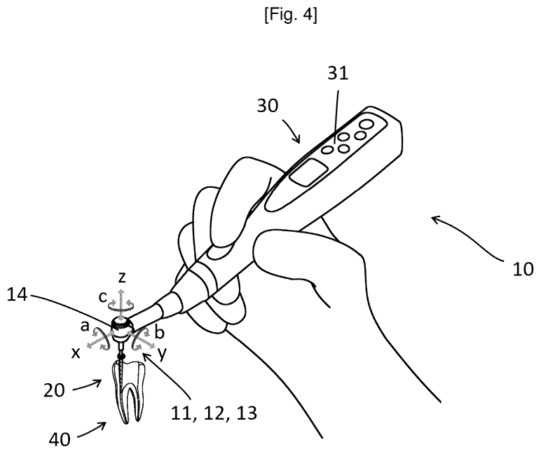

In order to have as much information as possible about the conditions of use of the instrument (20), the means (13) for measuring are preferably configured to measure the stresses experienced by the instrument (20) according to three axes of an orthonormal reference point. In reference to FIG. 4, it is a matter of capturing the efforts (x, y, z) and the torques (a, b, c). It is thus possible to completely obtain the load pattern experienced by the instrument (20).

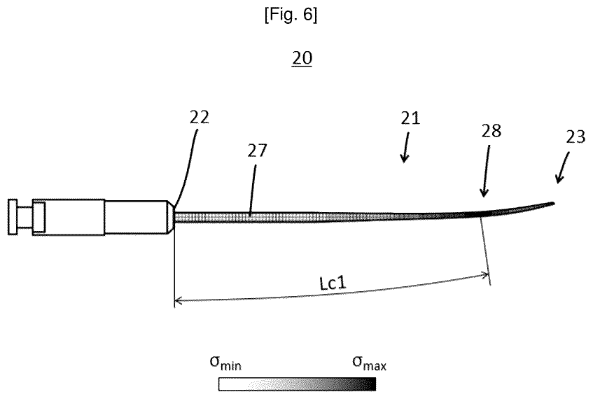

In reference to FIG. 6, the knowledge of the configuration in which the instrument (20) is modelled by the canal (42) at each moment, and the mechanical stresses it experiences, makes it possible to carry out a post facto analysis, using the finite-element method, of the effective distribution of the mechanical stresses within the instrument (20).

This analysis takes into account the design of the instrument (20), i.e. the material composing it, its nominal shape such as its dimensions and its cutting lips, and any treatments the instrument (20) has undergone upon manufacture, for example heat treatment.

The subdivision of the instrument (20) into a network (27), the knowledge of the load pattern to which the instrument (20) is subjected, and the choice of conditions at the extremes based on the acquisition of the shape of the canal (42), make it possible to calculate how the stresses are distributed within the instrument (20), and particularly to know the zone(s) of maximum stress (28) at the chosen given moment. On FIG. 6, the zone of maximum stress (28) is located on a first length (Lc1).

The working depth (Lp) and the stresses evolve during the treatment.

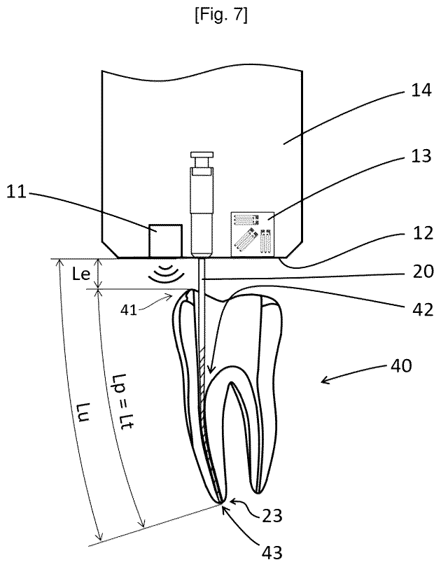

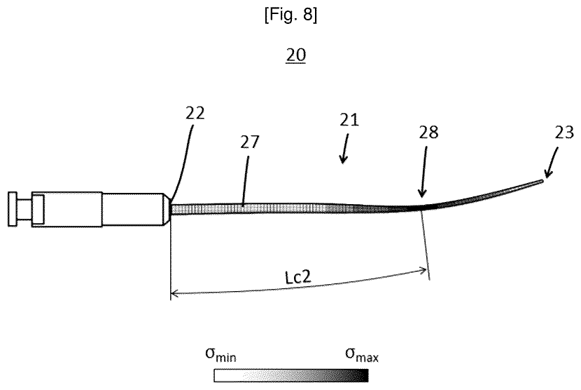

On FIG. 7, the tip (23) of the instrument (20) reaches the apex (43). The canal (42) models the instrument (20) according to a certain configuration, and we can see on FIG. 8 that the zone of maximum stress (28) has moved to a second length (Lc2), shorter than the first length (Lc1).

The recording in real time of the working depth (Lp) of the instrument (20) and the stresses it experiences makes it possible to carry out post facto a number of finite-element analyses, enabling the designers of endodontic materials to best design the material and its conditions of use.

On the basis of the calculations performed:
the manufacturer of the instrument (20) can modify the shape, the material or the treatments of the instrument (20);
the manufacturer of the instrument (20) can modify the planned conditions of use, such as the speed of rotation of the instrument dynamic;
the manufacturer of the handpiece (10) can integrate additional safety functions into the computer program run by the control unit (30).

In order to provide a sufficient database for the material manufacturers, the program of the control unit (30) is programmed to record at regular intervals, for each treatment:
the working depth (Lp) of the instrument (20);
the mechanical stresses experienced by the instrument (20).

The intervals can be a distance, for example a step of 0.5 mm. The database therefore contains a recording of the conditions of use of the instrument (20) in each configuration imposed by the shape of the canal. The size of the database is limited.

The intervals can be a duration, for example a step of 0.5 s. The database is larger because the practitioner makes back and forth movements during the treatment: there are therefore several recordings for a given working depth (Lp), but the recordings are much more complete and make it possible to better monitor the evolution, at each moment, of the conditions of use of the instrument (20).

The recordings also include:
the reference of the instrument (20) used, which makes it possible to obtain the characteristics necessary to the finite-element analysis, by consulting an additional database comprising the mechanical characteristics of the instruments (20) designed to be used in cooperation with the handpiece (10);
the acquisition of the shape of the canal (42), which is preferably a three dimensional acquisition so the finite-element analysis is as relevant as possible.

The records can be accompanied by additional data acquired by the control unit (30), of example moments where the irrigation of the canal (42) was or was not activated, with irrigation allowing evacuation of the debris and lubrication of the blade of the instrument (20). They may also be parameters of the instrument dynamic, such as the speed of rotation, the reciprocal angles, or the torque exerted by the motor.

These recordings can also be accompanied by data entered by the practitioner, preferably by means of the control unit (30) or by means of a computer connected to the database of recordings. The practitioner may for example declare whether an instrument breakage occurred, so the post facto analysis can be targeted at treatments having led to breakage.

With the aim of automating the process, the breakage of the instrument (20) is detected automatically by the computer program, for example by identifying a discontinuity of measurement of the stresses: a sudden release of the stress means that the instrument (20) has broken.

The analysis of several successive recordings, incrementally, makes it possible to reconstitute how the treatment took place and to identify, or at least suspect, the causes having led to the breakage. This may be misuse of the instrument (20) if the practitioner has used an inappropriate instrument dynamic or insufficient irrigation.

The shape of the canal (42) is of course the major criterion leading to instrument breakage. The post facto analysis of a large number of treatments carried out makes it possible to identify, for treatments having common parameters, the conditions having led to the breakage, or conversely having led to the success of the treatment.

The common parameters are of course the use of the same model of instrument (20) and a similar shape of the root canal (42).

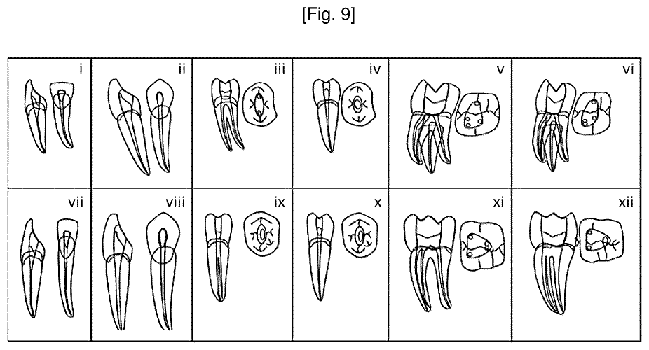

In reference to FIG. 9, different types of shape (i-xii) of canals (42) are illustrated. Each shape (i-xii) presents a treatment difficulty of varying complexity. For example, the profile (x) is straight and does not present any particular difficulty. However, the profile (ix), although generally straight, presents a bifurcation at which the practitioner must ensure he does not direct the instrument (20) to the wrong extension.

The database of recordings preferably comprises recordings from the largest possible number of practitioners, in order to have a sufficient population of data from different treatments, and particularly different shapes of canals (42). The analysis performed from the recordings is therefore more complete, which limits the impact of certain usage biases.

These usage biases can for example be a practitioner who tends to always prefer a first model of instrument (20) for a given treatment type and shape of canal (42), when another practitioner prefers another model of instrument (20). Analysis of the recordings makes it possible to choose the best model of instrument (20), the best instrument dynamic, or the most appropriate approach.

The constitution of the database of recordings has two purposes:
a first use is to enable new ongoing improvement actions by the material manufacturers;
a second use is to be able to identify, for a tooth (40) to be treated, which will be the high-risk zones, how to address them so the treatment is a success, and what conditions would very likely lead to instrument breakage.

On the basis of the acquisition of the shape of a canal (42) to be treated, the computer program is programmed to identify within the database which profile (i-xii) is the closest match, consequently which high-risk zones require vigilance by the practitioner, and potentially adaptation of the approach or the instrument dynamic.

This advance analysis makes it possible to guide the practitioner during the treatment.

When the practitioner performs the treatment, the depth gauge (11) detects at what working depth (Lp) the instrument is. If it proves that the working depth (Lp) is close to the depth of a high-risk zone, the practitioner is alerted to it so he can take the necessary precautions.

For that purpose, the control unit (30) activates the warning means of the handpiece (10). This may be an audible warning, a haptic warning such as a vibration, or more simply a visual warning displayed by the interface (31).

A colour code may be associated, for example to three risk levels:
a first colour, for example green, means that the instrument (20) is not at risk of breakage;
a second colour, for example yellow, means that there is a medium likelihood of risk of breakage, and vigilance by the practitioner may be sufficient;
a third colour, for example red, means that the likelihood of occurrence is high and an adaptation is imperative.

Advantageously, the computer program also takes into account the level of mechanical stresses measured by the means (13) of measuring, so as to refine the analysis and guide the practitioner more precisely: for example, it is pointless to warn the practitioner unduly if he is in a high-risk zone but the necessary adaptations have been implemented.

Lastly, the computer program can be programmed to automatically adapt the dynamic of the motor driving the instrument (20) if it detects that the likelihood of breakage is too high: the speed of rotation can be reduced, or the drive can be stopped.

Additionally, the computer program is programmed to be able to adapt, through artificial intelligence, the teachings arising out of the analysis of the previous recordings to a new shape of canal (42) or to a new reference of instrument (20).

For example, a change of material of the instrument (20) can be taken into account and post facto analyses can be recalculated on the basis of the new parameters to obtain a simulation of what such change would lead to. The same applies for changes to the shape of the instrument (20), and more generally for any influential parameter.

Or also, if a canal (42) presents for example a sharp bend or a bifurcation at a different depth to what is known within the database, the computer program is programmed to nevertheless identify this critical zone for the guidance of the practitioner to be effective and efficient during treatment.

Furthermore, the method and the handpiece (10) can be modelled differently from the examples given without departing from the scope of the invention, which is defined by the claims.

In particular, the control unit (30) covers generally any electronic equipment used for the implementation of the methods and the handpiece (10) described. It is therefore a microcomputer integrated into the handpiece (10), but also any computer or smartphone used by the practitioner and able to run the computer program, or upload or download the recordings of the database.

The term “computer program” is also to be interpreted within a broad sense, and covers the sub-programs and additional functions implemented in the methods described. Non-exhaustively, they are:
a program driving the motor of the handpiece (10);
the program for acquiring data measured by the depth gauge (11) and the means (13) for measuring, as well as their recordings;
the program of post facto analysis by finite-element method of the conditions of use of the instrument (20) in the different configurations;
the program of analysis of the recordings making it possible to establish the profiles of the teeth (40) and canals (42) to be treated, including the corresponding critical zones;
the program of advance analysis of critical zones of a canal (42) to be treated.

In a simpler embodiment, it is the practitioner who identifies which are the high-risk zones, without using the database. For that purpose, the practitioner uses the acquisition of the shape of the canal (42) to be treated, and enters the depths of the high-risk zones on the basis of his own analysis. Upon treatment, the handpiece (10) is able to warn the practitioner when he approaches a high-risk zone, on the basis of the measurements taken by the depth gauge (11).

Furthermore, the technical features of the different embodiments and variants mentioned above can be, in their entirety or just for some of them, combined with each other. Thus, the method and the handpiece (10) can be adapted in terms of cost, functions and performance.

1-9. (canceled)

10. A method for collecting usage data of an endodontic instrument, remarkable in that it comprises the following steps:
acquiring data relating to the shape of a root canal to be treated;
at regular intervals, acquiring data relating to the working depth of the instrument in the root canal;
at regular intervals, acquiring data relating to stress undergone by the instrument during use; and
producing a database linking each data item acquired.

11. The method for collecting data according to claim 10, wherein the data are acquired at regular intervals of a predefined duration.

12. The method for collecting data according to claim 10, wherein the acquisition of data relating to the shape of the canal is a three-dimensional acquisition.

13. The method for collecting data according to claim 10, wherein data of the instrument dynamic are also recorded within the database.

14. The method for collecting data according to claim 10, wherein additional data, such as the state of activation or inactivation of the irrigation, are also recorded within the database.

15. The method for collecting data according to claim 14, wherein the additional data comprise information about the breakage or otherwise of the instrument during the treatment.

16. The method for collecting data according to claim 10, wherein the data relating to stress are a complete torsor at a given point of the stress experienced by the instrument, obtained by measuring the stress according to three axes of an orthonormal reference point.

17. The method for collecting data according to claim 10, further comprising a step of finite-element calculation based on:
the shape of the canal;
a shape and mechanical properties of the instrument;
the working depth of the instrument at a given interval; and
the stress at that same given interval;
in order to determine the local stresses within a material constituting the instrument for the recording at the given interval.

18. A handpiece for endodontic practice, comprising a control unit running a computer program, and designed to drive a root canal instrument, wherein the handpiece comprises means of detecting a distance between a reference point and a part of a tooth, and means for measuring mechanical stresses experienced by the root canal instrument, connected to the control unit, wherein the computer program is programmed to determine a depth at which the instrument is during use according to a known length of the instrument, and the mechanical stresses experienced by the instrument during use, and wherein the computer program is configured to upload into a database the data acquired relative to the mechanical stresses and the depth.
